(19) United States
(12) Patent Application Publication
(10) Pub. No.: US 2010/0133103 A1
(43) Pub. Date: Jun. 3, 2010

(54) METHOD AND A DEVICE FOR MONITORING PHYSICAL CONDITIONS

(75) Inventors: Lars Ericsson, Malmo (SE); Berit Stjernfeld, Malmo (SE)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: COOLGUARD AB, MALMO (SE)

(21) Appl. No.: 11/568,839

(22) PCT Filed: May 4, 2005
(86) PCT No.: PCT/SE05/00648

§ 371 Date: Feb. 6, 2008

(30) Foreign Application Priority Data
May 12, 2004 (SE) 0401224-1

(51) Int. Cl. G06F 15/16; G01N 33/02; G01N 27/26

(52) U.S. Cl. 204/406; 426/231; 709/219

(57) ABSTRACT

A method for monitoring the physical conditions of products, such as food-stuff and similar, wherein a plurality of detectors associated to nodes (25; 31) is arranged close to the food-stuff. A detector (26; 34) continuously measures at least one physical condition of a product. The nodes (25; 31) establish continuously and periodically a wire-less contact with the central unit (12), said nodes and said central unit being part of a local installation, and also transfer measured data from the detectors (26; 34) to the central unit (12). The central unit (12) continuously stores the received measured data and continuously and periodically establishes a wireless contact with a server (11) connected to the Internet for the transfer of stored measured data. Information related to the transferred measured data is made available on the server (11) to be reviewed through the Internet. The central unit (12) comprises a first communication means (14) for a wireless communication with a web server (11) connected to the Internet and a second communication means (16) for a wire-less communication with the nodes. The nodes comprise a communication means (17) corresponding to the second communication means for having a wire-less communication with the second communication means. Furthermore, the nodes comprise detectors (12; 34) and detector elements (27; 39) for recording data relating to the physical condition of the object. The nodes are arranged to continuously and periodically establish a wireless contact with a central unit (12) comprising a storage unit (53) for storing a plurality of measured data from a plurality of nodes.

### TECHNICAL FIELD

The device relates to a device for monitoring physical conditions of objects. Specifically the invention can be applied in connection with monitoring the temperature of cold or frozen food-stuffs. Also other physical conditions such as pressure and humidity can be monitored in accordance with the invention.

### PRIOR ART

The temperature and other physical conditions of for instance food-stuffs are monitored more and more careful so as to ensure that the quality and conditions can be kept above a specified level. In refrigerated counters an frozen-food displays there are provided temperature detectors which in different ways are capable of producing an alarm or indicate in other ways that some kind of limits have been exceeded.

General equipment for measuring the temperature is disclosed in GB2331581.

The equipment is mobile and comprises temperature detectors for contact measuring and non-contact measuring. A laser sight is provided for the non-contact measuring.

Other systems exist for a direct measuring of the temperature of food-stuffs. JP2001050820 discloses a temperature measuring device that can be disposed together with the food-stuffs in a space. A calculating and storing unit is enclosed beside the temperature measuring device. The temperature in spaces can be measured continuously and histograms and other data can be determined.

The measuring processes that are carried out in accordance with the techniques described above will provide point by point information about the temperature of objects or food-stuff and to some extent will allow a compilation of information. However, a drawback is that information is not easily available. The technique also has shortcomings in relation to the possibilities to monitor.

### SUMMARY OF THE PRESENT INVENTION

An object with the invention is to overcome the drawbacks and shortcomings mentioned above and to provide a method for monitoring physical conditions, particularly of food-stuff. It is also an object to provide a device allowing and efficient monitoring of the physical condition and a continuous provision of information that has been gathered.

The object is achieved by disposing detectors in or close to the goods or products that will be monitored. The detectors are formed to communicate wirelessly with a central unit and to transfer recorded measured data to it. Received measured data are continuously stored in the central unit. The central unit will establish a wireless contact with a server and will transfer measured data to it. The server is connected to the Internet and is designed to make available information that is related to the recorded measured data through the Internet.

The detectors that are used in accordance with the invention are stand alone devices and are connected to the central unit through a wireless connection. A detector element having a temperature sensor or another corresponding sensor associated to the detector can be disposed at some distance from the detector and very close to the goods. The sensor can be a conventional type sensor and can be connected to the detector by wire. The detector comprises also means for a wireless connection with the central unit, which is provided in the same premises as the detectors or close to these premises. Preferably radio is used for this connection. The detector is arranged to establish independently the connection with the central unit and to transfer recorded measured data during a communication phase.

The central unit is provided with corresponding means for a wireless connection with the detectors and with memory means for a continuous storing of the measured data that have been received from the detectors. The wireless connection will allow a communication at least within the premises. The information consisting of or related to the received measured data is further transferred to a server which can be arranged at a large distance from the central unit and the detectors. It is possible to use a cellular telephone system such as GSM, GPRS or so called 3G in this communication. If a conventional cellular telephone system is used measured data can be transferred by so called SMS (Short Message Service).

It is possible also to provide the central unit with further interfaces, such a conventional interface to a computer or a so call web interface, for setting up and for performing service of the central unit. The central unit also comprises memory means for storing measured data.

### BRIEF DESCRIPTION OF THE DRAWINGS

The invention will be explained in more detail below with reference to the accompanying drawing. It should be noted that the drawings show examples of embodiments in accordance with the invention.

FIG. 1 is a block diagram of an installation designed in accordance with the invention and

FIG. 2 is a perspective view schematically showing an embodiment of a hand unit used in the installation of FIG. 1.

### THE INVENTION

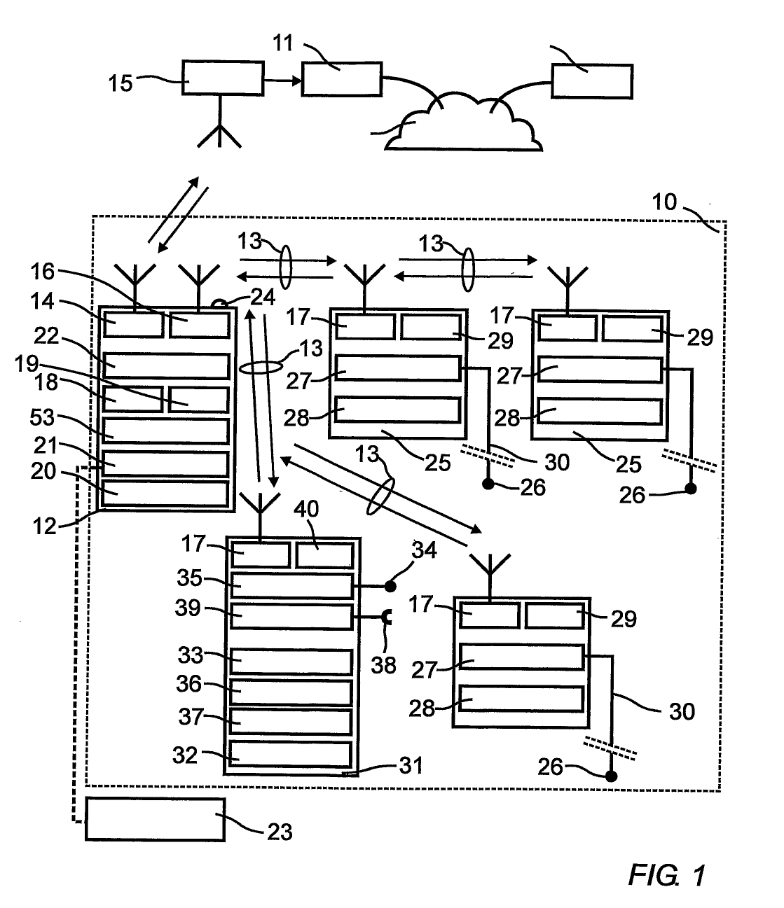

In the embodiment of a system in accordance with the invention shown in FIG. 1 a local installation 10 is connected to a web server 11 through a wireless connection. The wireless connection can be a wireless telephone connection, such as GSM, or another similar connection type. In the local installation there is provided a plurality of a set out units or nodes of at lest two different types and a central unit 12. The units of the local installation can be allocated in different rooms or adjacent premises. The notes are arranged in cold stores, frozen-food displays and similar spaces close to the goods, the temperature of which has to be monitored.

The central unit 12 is connected to the distributed units through a wireless connection, preferably a radio connection 13. In the central unit 12 there is provided a first communication means 14 for mobile or wireless telephony. The first communication means 14 is used for a wireless communication between the central unit 12 and the web server 11 through a cellular telephone arrangement 15 of a telephony provider. Preferably a built in antenna is used. In some applications an outlet for an external extra antenna is included. There is a communication established with the cellular telephone arrangement established at least each day. In this context the expression cellular telephone arrangement is used for any constructions that are established for a wireless telephone and data communication to and from public telephone networks and data networks.

The central unit 12 also comprises a second communication means 16 for a wireless communication with the nodes of the local installation. The second communication means 16 can include a transmitter and a receiver for radio signals within open radio bands, such as around 433 MHz. The nodes included in the local installation comprise a corresponding communication means 17.

In a conventional manner the central unit 12 can be provided also with a display 18, an input unit 19, such as a key pad, and a battery 20. The setting up and service of the central unit can be handled through a computer interface 21 and an external computer 23. The central unit is controlled by a controlled unit 22 which can comprise also memory means and other required components. The control unit can be arranged to communicate with the external computer 23 through a so called web interface. Such an interface will facilitate the setting up of the control unit and other management, such as safety backup. Preferably battery 20 is chargeable. An associated charging device can be embedded or arranged externally.

The communication between the first communication means 14 and the cellular telephone installation 15 is suitably handled through a message or data ex- change. In more simple applications it is possible to use SMS. An efficient communication can be achieved by the use of a specific protocol.

The control system of the central unit is designed to be able to communicate with approximately 256 nodes in a local installation. Measured data from the nodes are collected from a few times per hour to a few times per day. It is appropriate to use intervals of two collections each hour but also as often as twenty times per hour or more seldom such as collections every third hour may take place. In the memory means of the control unit there is space enough for storing data that have been measured during at least one week.

If a measured quantity lies outside acceptable intervals, or an error condition arise, different forms of alarms can be generated. Locally an alarm can be given by a flashing light 24. It is possible also to perform an alarm transmission. Normally a unit producing the alarm has to be reset before a new alarm can be produced in the same unit. It is possible also to reset automatically if the course of the alarm is eliminated.

Quantities outside predetermined intervals can be allowed for some time and at some conditions, such as when deep freeze equipment is defrosted. After the time period accepted for such a condition an alarm will be given.

The central unit 12 stores, besides measured quantities, different data about events, error conditions and activities. These data are available through the computer interface or by other means for trouble shouting and service. The central unit 12 is designed to handle different categories of personnel. Activated alarms can be reset by a first category of personnel. Normally a higher security or authorization check is not required for resetting the central unit 12.

An authorizing code of a first level is required when a node is switched off from the local installation. The same level can be applied in connection with an exchange of the name of a node and similar measures. The authorization code can be input through the input device 19. An authorization code of a second higher level is required for more qualified measures, such as the setting up of parameters of nodes or the central unit and for reading error reports and similar information have been stored in the central unit.

FIG. 1 also shows a first node type 25 comprising a first detector 26. In a plurality of applications the first detector is a temperature detector but also other detectors such as for measuring pressure, existence or non-existence of a gas or light also exist in some applications. The node 25 comprises detector elements 27 for the detector 26 and a battery 28 for a relatively extended working period. The working period can be up to several years. The detector 26 can be connected to the detector element 27 through a measuring cable 30 or in some applications through a further wire-less connection. As a result the complete node does not have to be in direct contact with any measuring object or be disposed in a measuring environment.

The node 25 will regularly establish a wireless communication with the central unit 12 through a communication means 17. The communication means 17 of the node corresponds to the second communication means 16 of the central unit 12. A controlled unit 29 of the node is operatively connected to other units of the node for controlling them. Each node is provided with a unique address or a unique ID number. Preferably the ID number is visible outside a housing or a casing protecting the electronic components of the node. When a node is installed in a local installation the node is provided also with a virtual number or name used for identification at a superior level of the web server 11 and other equipment. The unique address is transferred together with measured data to the central unit 12 and is stored in a storing unit 53 of the central unit.

The communication means 17 of the node is arranged to repeat signals from other nodes. As a result nodes at a larger distance from the central unit 12 still are able to contact the central unit. Preferably the control unit 29 of the node is instructed by the central unit to enter a repeating mode in parallel with the normal communication mode with the central unit. Nodes that have a repeating function can be provided with an extended battery capacity or be provided with an external power supply so as to be active during longer continuous time periods.

When a node 25 is installed it will immediately establish contact with the central unit 12. As soon as contact has been established the node will indicate this to an operator for instance by a light or sound signal. The node will be assigned the virtual number by the central unit and this number will be used in the future during further communication with the web server. The operator can also see to that the virtual number or another name of the node will reflect the function and/or location of the node to make it more simple to relate to the node in the local installation. The detector 26 and the detector element 27 are provided for measuring in the temperature interval of −30−+70 (±1) ° C.

A hand unit 31 can be included as a second node type in a local installation 10. The hand unit 31 is provided with a battery unit 32 which preferably is chargeable and a memory unit 33 for storing a plurality of measured values, preferably at least 250 values. A temperature detector 34 is connected through a cable to a detector element 35 of the hand unit. The temperature detector and the detector element are functionally comparable to corresponding means of other nodes.

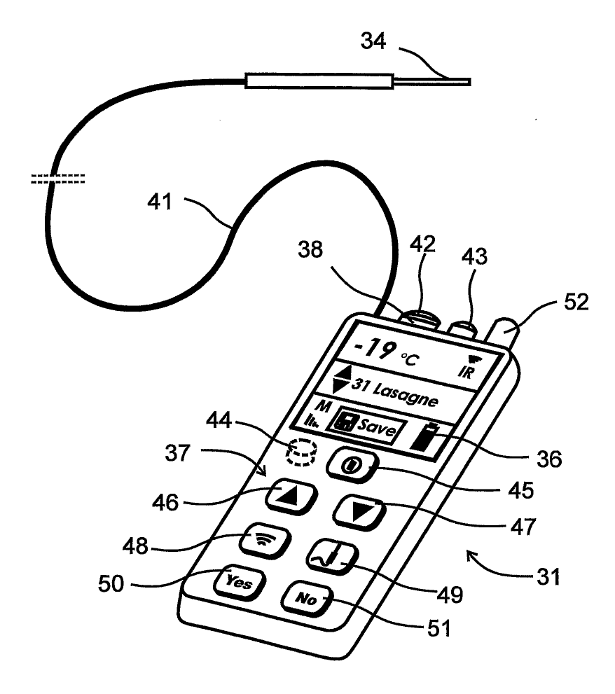

The hand unit is easily handled because of the provision of a display 36 and an input unit in form of a key pad 37. These means are described further below with reference to FIG. 2.

Unlike other nodes the hand unit comprises also a second detector means formed as a non-contact detector 38. In the embodiment shown the non-contact detector is an IR temperature sensor capable of sensing the temperature of objects at some distance. The non-contact detector 38 is connected to a measuring means 39 of the hand unit. A control unit 40 controls and monitors the function of the units included in the hand unit.

An embodiment of the hand unit 31 is shown in FIG. 2. In this embodiment the display 36 has a rectangular shape and comprises an essentially rectangular display surface in a casing enclosing the hand unit. The key pad 37 in this embodiment comprises seven buttons with different functions. The temperature detector 34 is connected to the measuring means embedded in the casing through a measuring cable 41. The display shows in plain text the goods or products involved in a measuring process. The names of the goods are transferred initially from the central unit and will remain locally stored in the hand unit. Name and other properties of new goods are put in through the central unit.

In this embodiment the non-contact detector 38 comprises a lens 42, so as to decrease the measuring angle. In one embodiment the lens is a fresnel lens having a refractive index 10.2 with a diameter of 22 mm and the thickness 0.5 mm. The active surface of the detector is approximately 1.2 mm in diameter and together with other dimensions the result is a measured surface of approximately 140 mm in diameter on an object, if the hand unit is situated 1 meter from the measured object. A laser pointer 43 of conventional type is used to direct the hand unit when an object is measured.

A buzzer 44 embedded in the hand unit can be used for producing signals to an operator or user. The buzzer can be mounted directly on a circuit board also carrying other components and circuits of the hand unit.

The key pad 37 also comprises a plurality of buttons each having a clear function. A first button 45 is used to activate or switch on the hand unit. The step of activating includes switching on a background light of the display. Browsing among the goods that are included in the measuring process can be controlled by means of a second button 46 and a third button 47, which in the shown embodiment are marked with an arrow symbol. Suitably the designation of the goods as well as an identification number associated to the goods are shown when the operator browses.

Before commencing a measuring process the operator determines which detector that will be used during the measuring process. A fourth button 48 is used if the operator chooses IR, that is a non-contact measuring, and a fifth button 49 is used by the operator to choose a measuring process using a wired detector. In the event a question is shown on the display or when selections are possible a sixth button 50 is used to give the response yes or to accept, and a seventh button 51 is used to respond with a no or to not accept. There is an indication on the display of an on-going measuring process and when this has come to an end. The indication can be in form of a symbol or present temperature value flashing during the measuring process and a sound and/or light signal is given when the measuring process has come to an end. Then it is possible for the operator to know that a new measuring process can start. The communication means 17 provided in the hand unit in the shown embodiment is connected to an antenna 52 for communicating with the central unit.

A synchronization in time between the central unit and the nodes associated to it and a required adjustment with regard to daylight saving can be handled through SMS messages. A central unit can on a regular basis or after an input transmit a SMS message to itself. A time stamp in the SMS message can be interpreted and be used for the adjustment of a clock unit in the central unit. Synchronization and time adjustment of nodes is achieved when the nodes repeatedly establish contact with the central unit and from the central unit receive information about the present time.

The central unit 12 will at a regular basis receive data from the nodes associated to it and will record the actual time for the reception. If data fail to appear from an associated node an alarm is given in an appropriate way. The alarm signal can be given locally as well as by transmitting a message to the web server. The central unit can be arranged to allow a certain number of failed contacts from a node before an alarm is given. Alarm messages of different kinds can also be transmitted to another external unit besides the web server, such as a specifically arranged server. Such alarm messages suitably are transmitted through a second web server or similar unit through a cellular telephone installation in a similar way to what was described above.

1. A method for monitoring the physical condition of products such as food-stuff and similar, wherein a plurality of detectors associated to nodes (25; 31) are arranged close to the food-stuff, characterized by
measuring continuously in a detector (26; 34) at least one physical condition of a product,
the nodes (25; 31) continuously and periodically establishing a wireless contact with a central unit (12), said nodes and central unit being part of a local installation,
the nodes (25; 31) transferring measured data from the detectors (26; 34) to the central unit (12),
the central unit continuously storing the received measured data,
the central unit (12) continuously and periodically establishing a wireless contact with a server (11) connected to the Internet and transferring stored measured data, and
presenting on the server (11) information related to the transferred measured data to be available over the Internet.

2. A method in accordance with claim 1, also including the step of
the central unit (12) continuously recording the actual time when a node transfers measured data and transmits an alarm signal when an expected transfer fails to appear.

3. A method in accordance with claim 1, also including the step of the central unit (12) comparing measured data transferred from the nodes with intervals for the measured values stored in advance and transmitting an alarm signal when the measured data differ from the intervals.

4. A method in accordance with claim 3, also including the step of forwarding the alarm signal to the server (11).

5. A method in accordance with claim 1, also including the step of
repeating in a first node type signals from other nodes in the local installation to the central unit.

6. A method in accordance with claim 1, also including the step of
providing each node in the local installation with a unique identity designation.

7. A method in accordance with claim 1, also including the step of
performing a none-contact measuring with a second node type, said second node type continuously indicating an ongoing measuring process.

8. A method in accordance with claim 1, also including the following steps:
transferring at least one designation associated to each product to the hand unit (31) and
displaying the designation at a display (36) based on the activity of an operator.

9. A device for monitoring physical conditions of products, such as food-stuff and similar, wherein a plurality of detectors associated to nodes (25; 31) is arranged close to the food-stuff and wherein the nodes are comprised in a local installation, characterized in
that the local installation comprises at least one central unit (12) and a set of nodes of a first node type,
that the central unit (12) comprises a first communicating means (14) for a wireless communication with a web server (11) connected to the Internet,
that the central unit (12) comprises a second communication means (16) for a wireless communication with the nodes,
that the nodes comprise a communication means (17) corresponding to the second communication means for a wireless communication with the second communication means,
that the nodes comprise detectors (12; 34) and detector elements (27; 39) for recording data regarding the physical condition of an object,
that the nodes are arranged to continuously and periodically establish a wireless contact with a central unit (12) and
that the central unit (12) comprises a storing unit (53) for storing a plurality of measured data from a plurality of nodes.

10. A device in accordance with claim 9, wherein each node is provided with a unique identity designation.

11. A device in accordance with claim 11, wherein at least one node is constituted by a hand unit (31) and wherein said hand unit (31) is provided with a display (36) for indicating the measured object and a measured value, a memory unit (33) for storing a plurality of measured values and a temperature detector (34) connected through a measuring cable (30).

12. A device in accordance with claim 11, wherein the hand unit (31) comprises means (38) for a non-contact measuring of temperature and a key pad (37) for selecting either the temperature detector (34) or the means (38) for a non-contact measuring.

13. A device in accordance with claim 12, wherein the hand unit (31) comprises a light pointer (43) cooperating with the means (38) for a non-contact measuring.
